(19) United States
(12) Patent Application Publication
(10) Pub. No.: US 2020/0027534 A1
(43) Pub. Date: Jan. 23, 2020

(54) DEVICE, SYSTEM, AND METHOD FOR UPDATING PROBLEM LISTS

(71) Applicants: KONINKLIJKE PHILIPS N.V., EINDHOVEN (NL); UNIVERSITY OF CHICAGO SCHOOL OF MEDICINE, Chicago, IL (US)

(72) Inventors: Paul Joseph CHANG, Chicago, IL (US); Merlijn Sevenster, Haarlem (NL); Thomas Andre FORSBERG, Hayward, CA (US)

(21) Appl. No.: 16/337,985

(22) PCT Filed: Oct. 17, 2017
(86) PCT No.: PCT/EP2017/076514

§ 371 Date: Mar. 29, 2019

Related U.S. Application Data
(60) Provisional application No. 62/408,937, filed on Oct. 17, 2016.

(51) Int. Cl. G16H 10/60; G06F 16/93

(52) U.S. Cl. CPC G16H 10/60; G06F 16/93

(57) ABSTRACT

A device, system, and method updates a problem list. The method performed at a reporting server includes receiving a first document, the first document associated with a second document, the first document including first information. The method includes extracting first concepts from the first information of the first document. The method includes determining select ones of the first concepts to be included in the second document. The method includes updating the second document based on the select ones of the first concepts.

### BACKGROUND INFORMATION

A problem list (PL) provides information that specifies a clinical history of a patient. For example, the PL may include International Classification of Diseases (ICD) codes that is indicative of diseases, signs/symptoms, abnormal findings, complaints, external causes of injury/disease, etc. The PL may indicate whether a condition is no longer present or is currently ongoing as well as when the condition was diagnosed/treated. Thus, for various purposes such as radiological interpretation, the PL contains potentially relevant information if updated thoroughly and consistently. However, population of the PL is often laborious and conscientiousness in performing this task ranges between practitioners. Accordingly, this often results in incompletely and/or inaccurately populated PLs.

### SUMMARY

The exemplary embodiments are directed to a method, comprising: at a reporting server: receiving a first document, the first document associated with a second document, the first document including first information; extracting first concepts from the first information of the first document; determining select ones of the first concepts to be included in the second document; and updating the second document based on the select ones of the first concepts.

The exemplary embodiments are directed to a reporting server, comprising: a transceiver communicating via a communications network, the transceiver configured to receive a first document, the first document associated with a second document, the first document including first information; and a processor extracting first concepts from the first information of the first document, the processor determining select ones of the first concepts to be included in the second document, the processor updating the second document based on the select ones of the first concepts.

The exemplary embodiments are directed to a method, comprising: at a reporting server: receiving a first document, the first document associated with a second document, the first document including first information; extracting first concepts from the first information of the first document; generating a respective prompt for each of the first concepts to be displayed; receiving a respective input for each prompt; determining select ones of the first concepts to be included in the second document based on the prompts and the inputs; and updating the second document based on the select ones of the first concepts.

### BRIEF DESCRIPTION OF THE DRAWINGS

FIG. 1 shows a system according to the exemplary embodiments.

FIG. 2 shows a reporting server of FIG. 1 according to the exemplary embodiments.

FIG. 3 shows a method for updating a problem list according to the exemplary embodiments.

### DETAILED DESCRIPTION

The exemplary embodiments may be further understood with reference to the following description and the related appended drawings, wherein like elements are provided with the same reference numerals. The exemplary embodiments are related to a device, a system, and a method for updating a problem list (PL) of a patient. The PL may relate to a document or collection of clinical information of the patient. The exemplary embodiments provide a mechanism in which clinical documents of the patient are analyzed to determine the clinical information that is added to the PL in an automated manner or in a manual manner by prompting for an input.

The PL of a patient may be a document that describes and/or lists health related information of the patient that has occurred, was discovered, has been treated, or is still ongoing (e.g., diseases that have been contracted, injuries that have been suffered, etc.). Accordingly, when utilized and maintained properly, the PL may provide a clear picture of the health of a patient regardless of the professional reading the PL. As those skilled in the art will understand, the PL may provide various benefits including a personalized benefit such as aiding practitioners in providing customized care through identification of health related issues or a generalized benefit such as identifying disease-specific populations by analyzing PLs of a region where patients have a common health-related issue. Although PLs may be written and populated by a practitioner, the PLs may also be populated using encoded descriptions. Specifically, the PL may be populated with corresponding International Classification of Diseases (ICD) codes such as ICD-10 codes. The ICD-10 codes include over 14,000 different codes and may be used to track many diagnoses. Therefore, the ICD-10 codes may provide an exhaustive list of substantially all diagnoses that may be determined by practitioners.

It is noted that the description of the exemplary embodiments herein relates to the PL utilizing the ICD-10 codes. However, the use of the ICD-10 codes is only exemplary. In another exemplary embodiment, another version of the ICD codes may be used in the PL. In a further exemplary embodiment, a different type of encoding may be used such as SNOMED or RadLex. Thus, the use of the ICD-10 codes in the PL may represent any encoding scheme to represent a diagnosis.

The exemplary embodiments relate to updating the PL of a patient so that the PL is maintained in a substantially complete and consistent manner. Conventional implementations of populating the PL is often at the discretion of the practitioner who decides to include information or not. For example, a first practitioner may opt to be very conscientious by thoroughly reviewing a clinical document and include every relevant piece of information into the PL. In another example, a second practitioner may opt to use a less thorough approach and do a cursory review of a clinical document by only including a low percentage of potential pieces of information into the PL or including information that is of high importance only. Therefore, the reliance upon respective attitudes of the practitioner utilized in the conventional implementation results in inconsistent and incomplete population of the PL. In fact, surveys related to the reliability of the PL have been conducted and have shown mediocre results where only half of the practitioners surveyed would trust the information in the PL to be accurate and/or exhaustive.

Furthermore, regardless of the attitude of the practitioner, another issue arising in populating the PLs is a standardized definition of items that should and should not be included in the PL to maintain an accurate and updated PL. As noted above, as the practitioner is often left with the final decision to include or exclude a particular diagnosis. Accordingly, when different practitioners utilize the PL and/or when a plurality of PLs are stored in a repository for use by practitioners, many different styles of PLs may be included. In fact, there may be further considerations such as state and federal patient privacy requirements that may also impact what items should be included or excluded in the PL.

The exemplary embodiments are configured to update the PL by automatically determining potential information extracted from a clinical document. As will be described in detail below, the exemplary embodiments provide an automated manner in which the information is extracted from the clinical document. Therefore, the available information to be considered for inclusion in the PL is performed in a consistent manner. As will also be described in detail below, the inclusion or population of selected ones of the extracted information may also be performed using an automatic mechanism or a manual mechanism. When the inclusion operation is performed automatically, the inclusion operation is also performed in a consistent manner. Even when the inclusion operation is performed manually, the inclusion operation may increase the consistency as users are requested to respond to the extracted information which is an automated operation.

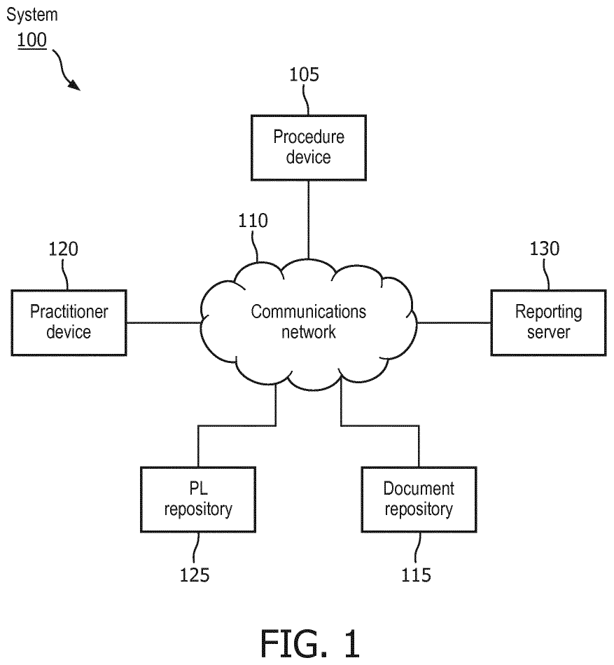

FIG. 1 shows a system 100 according to the exemplary embodiments. The system 100 relates to a communication between various components involved in updating a PL of a patient. Specifically, the system 100 may include a procedure device 105, a communications network 110, a document repository 115, a practitioner device 120, a PL repository 125, and a reporting server 130. As will be described in further detail below, the system 100 according to the exemplary embodiments incorporates an entirety of a process in which the PL of the patient is populated or updated.

The procedure device 105 may represent any electronic device that is configured to perform a procedure on the patient. For example, the procedure device 105 may be used for a radiological procedure such as an X-ray scan, a magnetic resonance imaging (MRI) scan, a computerized axial tomography (CAT) scan, etc. Accordingly, the procedure device 105 may include the necessary hardware, software, and/or firmware to perform the various procedures and/or treatments. Those skilled in the art will understand that the procedure device 105 may be configured to operate in an automatic manner, a manual manner, and a combination thereof. For example, the procedure may be performed by the procedure device 105 automatically from the user or technician initiating the procedure. Thereafter, the other parts of the procedure may be performed in an automated manner. In another example, the procedure may be performed by the procedure device 105 in which the user or technician must continuously provide inputs for actions to be taken in the procedure. The procedure device 105 may also include any connectivity hardware, software, and/or firmware for data to be communicated to another electronic device.

The procedure device 105 may accordingly generate results of the procedure for a user to read. For example, the procedure device 105 may generate images of tissue. The user who is reading the results may subsequently generate a medical document of the results of the procedure that was performed on the patient. For example, the user may utilize an application of the procedure device 105 or a separate device that is configured to generate the medical document. For exemplary purposes herein, it may be assumed that the procedure device 105 is configured to generate the medical document based on the inputs from the user. The medical document may be a document that lists or describes the results from the procedure being performed based on the inputs received from the user reading the results. For example, when the procedure device 105 performs an MRI scan in which fluid and tissue of the patient is represented in different shades of black, the user may read an image from the MRI scan and enter inputs for the medical document to be generated which describes shapes and sizes of various objects detected within images of the MRI scan. The medical document may also include or be associated with identification information. For example, a patient may be associated with an identification number to uniquely identify the patient. When the procedure device 105 is used for the patient, the resulting medical document may include or also be associated with the identification number.

It should be noted that the procedure device 105 generating images for a user to read is only exemplary. According to other exemplary embodiments, the procedure device 105 may generate other types of reports or results that is used in generating the medical document. The procedure device 105 being used for a medical procedure is also only exemplary. According to other exemplary embodiments, the procedure device 105 may represent any component that generates reports or results used in generating the medical document. For example, the procedure device 105 may be used to generate a surgery report, an interventional cardiology, a pathology report, an admission note, a progress note, a discharge note, etc.

It should also be noted that the system 100 illustrating a single procedure device 105 is only exemplary. Instead, the procedure device 105 may represent one or more procedure devices that are configured to exchange data with the other components of the system 100. For example, the procedure device 105 may represent a set of procedure devices 105 of a hospital that perform procedures and provide results used in generating medical documents.

It should also be noted that while the exemplary embodiments have been described as the procedure device 105 being related to medical imaging (e.g., MRI, X-ray, etc.), the procedure device 105 may be any type of device. Other examples of procedure devices include computed tomography (CT), positron emission tomography (PET), ultrasound, and hybrids such as PET-CT.

The communications network 110 may be configured to communicatively connect the various components of the system 100 to exchange data. The communications network 110 may represent any single or plurality of networks used by the components of the system 100 to communicate with one another. For example, if the procedure device 105 is used at a hospital or a private practice building, the communications network 110 may include a private network in which the procedure device 105 may initially connect. The private network may connect to a network of an Internet service provider to connect to the Internet. Subsequently, through the Internet, a connection may be established to other electronic devices. It should be noted that the communications network 110 and all networks that may be included therein may be any type of network. For example, the communications network 110 may be a local area network (LAN), a wide area network (WAN), a virtual LAN (VLAN), a WiFi network, a HotSpot, a cellular network (e.g., 3G, 4G, Long Term Evolution (LTE), etc.), a cloud network, a wired form of these networks, a wireless form of these networks, a combined wired/wireless form of these networks, etc.

The document repository 115 may be a component that stores medical documents. For example, the document repository 115 may be a database that maintains the medical documents in an electronic format. Accordingly, the procedure device 105 that generates the medical document may transmit the medical document via the communications network 110 for storage in the document repository 115. The document repository 115 may store the received medical documents in the database in a predetermined arrangement such as by identification numbers of the patients and/or by dates on which the medical documents were generated or the procedures were performed.

The document repository 115 may also include a search functionality that allows a user to query the document repository 115 to return at least one medical document based on an input entered for a search parameter. For example, a user may request that medical documents of a particular patient be returned. Accordingly, the identification number of the patient or the name of the patient may be provided as the search parameter to the document repository 115. In another example, a user may request a specific medical document or a set of medical documents of a particular patient be returned. Accordingly, the identification of the patient and a further search term (e.g., a specific procedure performed on a particular date at a particular time, any procedure performed in a selected period of time, etc.) may be provided as the search parameter to the document repository 115.

The practitioner device 120 may represent any electronic device that is configured to perform the functionalities corresponding to use associated with a healthcare provider. For example, the practitioner device 120 may be a portable device such as a tablet, a laptop, etc. or a client stationary device such as a desktop terminal. The practitioner device 120 may include the necessary hardware, software, and/or firmware to perform the various operations associated with medical treatment. The practitioner device 120 may also include the required connectivity hardware, software, and firmware (e.g., transceiver) to establish a connection with the communications network 110 to further establish a connection with the other components of the system 100. For example, the practitioner device 120 may schedule appointments for patients using a calendar application, may track treatments or procedures of a patient, etc.

As described above, the procedure device 105 may be configured to perform a procedure as well as generate a medical document that describes results associated with the procedure. In this manner, the medical document may be automatically generated and stored in the document repository 115. However, it should be noted that the procedure device 105 is only an exemplary source from which medical documents may be provided. In another example, the practitioner device 120 may also generate and transmit medical documents for storage on the document repository 115. Specifically, the practitioner may be a user of the practitioner device 120 who manually enters information into a form document or a free-form writing document to create the medical document. The user may include any pertinent information into the medical document including the identity of the patient. Subsequently, the medical document may be transmitted for storage in the document repository 115. In further examples of sources, the medical documents may be transported from other storage components or other sources that those skilled in the art will understand can provide a medical document.

In a substantially similar manner as the procedure device 105, the system 100 illustrating a single practitioner device 120 is only exemplary. Instead, the practitioner device 120 may represent one or more practitioner devices that are configured to exchange data with the other components of the system 100 via the communications network 110. For example, the practitioner device 120 may represent a set of practitioner devices used by practitioners of a hospital.

The PL repository 125 may be a component that stores PLs. For example, the PL repository 125 may be a database that maintains the PLs in an electronic format (e.g., an Electronic Medical Record (EMR)). In a substantially similar manner as the document repository 115, PLs for respective patients may be stored in the PL repository 125 for use by practitioners. It is noted that, typically, a patient may have only one associated PL that is stored in the PL repository 125. For example, a patient may have only one identification number and only one PL may be associated with this identification number. However, there may also be instances where a single patient has multiple associated PLs. Also in a substantially similar manner as the document repository 115, the PL repository 125 may include a query functionality in which a PL may be queried and returned to a user requesting a PL of a patient. For example, a practitioner using the practitioner device 120 may input an identification number of a patient and have the PL of the patient returned.

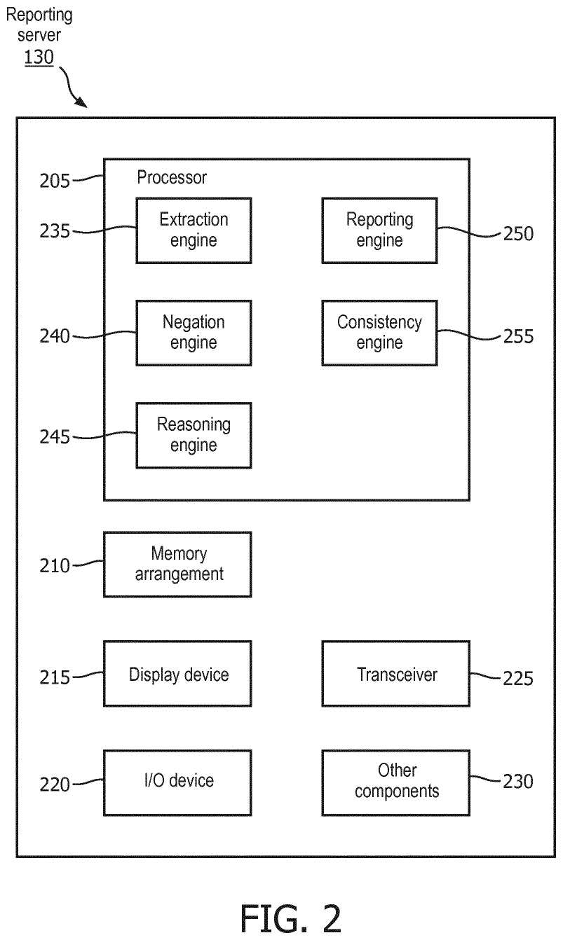

As described above, the reporting server 130 may be a component of the system 100. Specifically, the reporting server 130 may perform functionalities associated with updating the PL of the patient. FIG. 2 shows the reporting server 130 of FIG. 1 according to the exemplary embodiments. The reporting server 130 may provide various functionalities in updating the PL of the patient. Although the reporting server 130 is described as a network component (specifically a server), the reporting server 130 may be embodied in a variety of ways such as a portable device (e.g., a tablet, a smartphone, a laptop, etc.), a client stationary device (e.g., a desktop terminal), incorporated into the procedure device 105 and/or the physician device 120, etc. The reporting server 130 may include a processor 205, a memory arrangement 210, a display device 215, an input and output (I/O) device 220, a transceiver 225, and other components 230 (e.g., an imager, an audio I/O device, a battery, a data acquisition device, ports to electrically connect the reporting server 130 to other electronic devices, etc.).

The processor 205 may be configured to execute a plurality of applications of the reporting server 130. As will be described in further detail below, the processor 205 may utilize a plurality of engines including an extraction engine 235, a negation engine 240, a reasoning engine 245, a reporting engine 250, and a consistency engine 255. The extraction engine 235 may extract concepts from a medical document. The negation engine 240 may refine the concepts extracted by the extraction engine 235 by removing concepts that may be negated in the medical document. The reasoning engine 245 may further refine the concepts extracted by the extraction engine 235 by merging certain concepts. The reporting engine 250 may update the PL of the patient based on the resulting concepts. The consistency engine 255 may determine any inconsistency that may exist in the PL and address any detected inconsistency.

It should be noted that the above noted applications and engines each being an application (e.g., a program) executed by the processor 205 is only exemplary. The functionality associated with the applications may also be represented as components of one or more multifunctional programs, a separate incorporated component of the reporting server 130 or may be a modular component coupled to the reporting server 130, e.g., an integrated circuit with or without firmware.

The memory 210 may be a hardware component configured to store data related to operations performed by the reporting server 130. Specifically, the memory 210 may store data related to the received medical documents and/or PLs. The display device 215 may be a hardware component configured to show data to a user while the I/O device 220 may be a hardware component that enables the user to enter inputs. For example, an administrator of the reporting server 130 may maintain and update the functionalities of the reporting server 130 through user interfaces shown on the display device 215 with inputs entered with the I/O device 220. It should be noted that the display device 215 and the I/O device 220 may be separate components or integrated together such as a touchscreen. The transceiver 225 may be a hardware component configured to transmit and/or receive data via the communications network 110.

According to the exemplary embodiments, the reporting server 130 may perform various different operations to detect concepts extracted from a medical document. Initially, as described above, the extraction engine 235 may extract concepts from a medical document. Specifically, the extraction engine 235 may analyze the medical document to determine the information that is to be included in the PL of the patient. For example, the extraction engine 235 may parse a prose writing in the medical document and determine whether any term or phrase is indicative of a diagnosis. The extraction engine 235 may then determine the corresponding ICD-10 code. In this manner, the extraction engine 235 may extract a concept from the medical document. In another example, the extraction engine 235 may determine whether any ICD-10 codes are already present in the medical document. The extraction engine 235 may extract this code from the medical document.

It is noted that the extraction engine 235 may utilize any known identification mechanism to determine how to identify concepts in the medical document such as SNOMED, RadLex, ICD, etc. For example, the extraction engine 235 may be programmed with or have access to a plurality of ontologies (e.g., disease ontology) that describe consistent, reusable, and sustainable descriptions of health-related issues (e.g., diseases). Accordingly, using the identification mechanism, the concepts from the medical document may be properly extracted. Furthermore, depending on the encoding used in the PL, the extraction engine 235 may be configured to convert the extracted concepts to the corresponding encoding used in the PL. For example, if the extraction engine 235 utilizes SNOMED concepts, the extraction engine 235 may be programmed with or have access to a mapping table that maps the extracted SNOMED term to the corresponding ICD-10 code.

Those skilled in the art will appreciate that since the operations being performed are done in an automated manner with defined parameters particularly for what information is to be included and excluded, the extraction engine 235 operates in a consistent manner. As described above, an administrator of the reporting server 130 may input the defined parameters which dictate the automated manner in which the extraction engine 235 determines whether a piece of information in the medical document is a concept to be extracted for consideration of inclusion in the PL. Therefore, each medical document that is analyzed by the reporting server 130 utilizes the same manner of extracting the concepts from medical documents.

As described above, the negation engine 240 may refine the concepts extracted by the extraction engine 235 by removing concepts that may be negated in the medical document. Specifically, the negation engine 240 may detect if an extracted concept (e.g., an ICD-10 code) is negated in the medical document from which the concept was extracted. For example, the negation engine 240 may utilize “scope” detection mechanisms to detect if phrases from which a particular concept was extracted appears negated in that context. In another example, the negation engine 240 may utilize language parsing mechanisms to determine whether an extracted concept has an associated negative language. In a particular example, the medical document may include the ICD-10 code C10. The practitioner may have entered the code C10 to indicate that the patient does not have this diagnosis. For example, the medical document may have a positive column and a negative column, the code C10 being in the negative column. However, the extraction engine 235 may simply recognize the code C10 to be included in the medical document and therefore include this code C10 as an extracted concept when the code C10 is clearly to be excluded. Accordingly, the negation engine 240 may be configured to determine whether any extracted concepts are to be removed from consideration given what is included in the medical document from which the concept was extracted. In this manner, the negation engine 240 may refine the set of extracted concepts that the extraction engine 235 generated. It is noted that the actual removal of the negated concepts may be performed at a later time such as by the reporting engine 250. Accordingly, the negation engine 240 may be configured to mark the concepts that are to be negated.

As described above, the reasoning engine 245 may further refine the concepts extracted by the extraction engine 235 by merging certain concepts. Specifically, the reasoning engine 245 may establish if an extracted concept is a more general or a more specific to another extracted concept. Thus, when the set of extracted concepts includes a first concept and a second concept where the second concept is a more specific concept from the first concept, the reasoning engine 245 may determine that the first concept may be removed from consideration due to, for example, its redundancy with the second concept. The reasoning engine 245 may particularly be appropriate when the extracted concepts are or corresponded to ICD-10 codes. As those skilled in the art will understand, the ICD-10 codes are organized as an inverse tree where more general codes are located at the top and more specific codes are located at the bottom. For example, a range of codes C00 to C49 relates to malignant neoplasms of respiratory and intrathoracic organs. A specific code C34 within the range is more specific and relates to malignant neoplasms of the bronchus and lungs. In a further specificity, a code C34.90 relates to a malignant neoplasm of an unspecified part of an unspecified bronchus or lung. Using this hierarchy, it may be established whether one code is more generic than another. For example, if the set of extracted concepts include code C34 and code C34.90, the reasoning engine 245 may be configured to eliminate the code C34 as the code C34.90 establishes the more specific diagnosis. In this manner, the reasoning engine 245 may further refine the set of extracted concepts that the extraction engine 235 generated. More specifically, the reasoning engine 245 may further refine a refined set of extracted concepts that the extraction engine 235 generated and the negation engine 240 has first refined. It is noted that the refining operations of the negation engine 240 and the reasoning engine 245 may be performed serially as described above, serially in an opposite manner, or in parallel. Furthermore, the reasoning engine 245 may be utilized when the extracted concepts of the medical document are to be included in the PL of the patient. Specifically, the reasoning engine 245 may be configured to merge the extracted concepts from the medical document with concepts or information already included in the PL of the patient. It is noted that the actual merging of the concepts may be performed at a later time such as by the reporting engine 250. Accordingly, in a substantially similar manner as the negation engine 240, the reasoning engine 245 may be configured to mark the concepts that are to be merged. It is also noted that the hierarchical configuration of the ICD-10 codes is only an exemplary reason as to why the reasoning engine 245 performs the merging functionality. Those skilled in the art will appreciate that there are various other reasons to merge concepts that do not relate specifically to a generic/specific relationship.

In a more particular manner of the operation of the reasoning engine, the reasoning engine 245 may use a pairwise comparison operation. Specifically, each concept in the remaining set of extracted concepts may be compared to each remaining concept in the remaining set of extracted concepts. This comparison may be performed to determine if there is any concept that was extracted that is more generic than any of the remaining concepts that was extracted. This comparison may result in one of three outcomes. In a first outcome, for a selected concept, there may not be any other concept in the PL that is more specific although there may be one or more concepts in the PL that are more general. In this case, there may be value in including the selected concept in the PL as the selected concept gives more detail about a diagnosis for the patient. In a second outcome, for a selected concept, there may be at least one concept in the PL that is more specific. In this case, there may be little value in including the selected concept in the PL as there is another concept that provides more detail about a diagnosis for the patient. In a third outcome, for a selected concept, there may be no concept that is more specific or more general. In this case, there may be value in including the selected concept in the PL as there is no other concept that covers the diagnosis of the selected concept. Using the above pairwise operation, the reasoning engine 245 may generate a further refined set of extracted concepts to be considered for inclusion in the PL of the patient.

As described above, the reporting engine 250 may update the PL of the patient based on the resulting concepts. Accordingly, the reporting engine 250 may first query the PL repository 125 for the PL of the patient (e.g., using an identification number associated with the patient). The reporting engine 250 may subsequently provide a report creation and/or viewing environment in which extracted concepts are highlighted or noted for inclusion into the PL of the patient. Specifically, the reporting engine 250 may recognize which of the extracted concepts are to be included into the PL of the patient. For example, as will be described in detail below, the reporting engine 250 may provide an automatic approach or a manual approach to determine which of the extracted concepts are to be included into the PL of the patient.

Initially, the reporting engine 250 may determine which of the remaining set of extracted concepts are to be included into the PL of the patient. As described above, a medical document may have been analyzed by the extraction engine 235 to determine a first set of extracted concepts, the negation engine 240 may have determined a second set of extracted concepts by removing negated concepts within the medical document (e.g., the second set is a subset of the first set after the removal), and the reasoning engine 245 may have determined the remaining set of extracted concepts by merging generic and specific concepts from the medical document (e.g., the remaining set is a subset of the second set after the merging). When the remaining set of extracted concepts has been determined, the reporting engine 250 may again utilize the reasoning engine 245 to determine whether there are concepts to be merged between the information included in the PL and the remaining set of extracted concepts of the medical document. For example, the reasoning engine 245 may again utilize the pairwise comparison operation described above. Thus, if the concepts in the remaining set of extracted concepts represents a set of first concepts and the concepts in the PL of the patient represent a set of second concepts, the reasoning engine 245 determines if any of the first concepts is more generic than any of the second concepts and vice versa. In this manner, the reasoning engine 245 may determine if any of the first concepts from the medical document are to be included in the PL of the patient. Again, if one of the first concepts is more specific than one of the second concepts in the PL of the patient, the selected first concept may be considered for inclusion as the selected first concept provides more detail of the diagnosis. In this manner, the reporting engine 250 may determine select ones of the extracted concepts (hereinafter “final extracted concepts”) from the medical document that are to be included in the PL of the patient. It is noted that the reporting server 130 utilizing the reasoning engine 245 at two separate times is only exemplary. In another exemplary embodiment, the reasoning engine 245 may be utilized a single time such as after the PL for the patient is received to perform an overall merging operation.

The reporting engine 250 may accordingly utilize the final extracted concepts from the medical document to be included in the PL of the patient using an automatic approach or a manual approach. According to a first mechanism in which the reporting engine 250 performs the inclusion of the extracted concepts from the medical document into the PL of the patient, the reporting engine 250 may utilize the automatic approach. The automatic approach may entail including each of the final extracted concepts into the PL of the patient. Therefore, the above process may be utilized in removing one or more of the extracted concepts generated by the extraction engine 205 from the medical document (e.g., via the negation engine 240, via the reasoning engine 245 for concepts within the medical document, and/or via the reasoning engine 245 for concepts with the PL). The final extracted concepts may be included such that concepts may also be replaced or removed in the PL (e.g., when the final extracted concept is more specific to a concept in the PL). When the reporting engine 250 has updated the PL of the patient, the reporting server 130 may transmit the updated PL of the patient back to the PL repository 125.

It is noted that when the automatic approach is used, the reporting engine 250 may include visual features or annotations in the viewing environment. For example, a user may select that the automatic approach be used in updating the PL of the patient. After the reporting server 130 has performed the above process, the reporting engine 250 may include the visual indicia. As described above, the reasoning engine 245 may have different types of results from the pairwise comparison including a more specific result (an extracted code from the medical document is more specific than a code in the PL), a more generic result (an extracted code from the medical document is more generic than a code in the PL), and an independent result (an extracted code from the medical document is neither more generic nor specific than any code in the PL). These types of results may utilize different visual indicia. For example, the independent result may utilize normal type face, the more specific result may utilize a highlight, and the more generic result may utilize a lowlight. Thus, a user viewing the updated PL may see how the medical document affected the update of the PL.

According to a second mechanism in which the reporting engine 250 performs the inclusion of the extracted concepts from the medical document into the PL of the patient, the reporting engine 250 may utilize a manual approach. The manual approach may entail the reporting engine 250 prompting a user to select whether extracted concepts are to be included into the PL of the patient. In this regard, the reporting engine 250 may utilize various features, particularly to highlight or lowlight whether an extracted concept was to be removed by the negation engine 240 or merged by the reasoning engine 245 as was described above as the visual indicia included in the updated PL of the automatic approach. Thus, the user may be prompted with each of the final extracted concepts and each of the final extracted concepts may include the corresponding visual indicia to provide further information to the user as to a value of including or excluding the final extracted concept. When the reporting engine 250 has completed the prompting process and received an input for inclusion or exclusion for each of the final extracted concepts, the reporting engine 250 may update the PL of the patient accordingly. Thereafter, the reporting server 130 may transmit the updated PL of the patient back to the PL repository 125.

The reporting server 130 may include further features that may improve the manner in which the PL of the patient is updated. As described above, the reporting server 130 may utilize the extraction engine 235, the negation engine 240, the reasoning engine 245, and the reporting engine 250 to provide a consistent manner in which the PL of the patient is updated. The reporting server 130 may further include the consistency engine 255 to provide a further operation in the updating of the PL of the patient. The consistency engine 255 may determine any inconsistency that may exist in the PL and address any detected inconsistency. Specifically, the consistency engine 255 may detect if there is a semantic discrepancy among a given medical document, the extracted concepts, and/or the PL of the patient. The consistency engine 255 may particularly be utilized when the manual approach is utilized in the reporting engine 250 (e.g., overlooked inconsistencies) and/or while analyzing the information in the PL of the patient.

In a particular example, the consistency engine 255 may utilize the extracted concepts that were removed via the negation engine 240. Although the extracted concepts removed by the negation engine 240 may be eliminated from consideration prior to updating the PL of the patient (in the automatic approach), the extracted concepts removed by the negation engine 240 may provide further insight as to whether an incorrect entry or diagnosis is present in the PL of the patient. For example, the negation engine 240 may have determined that the medical document negated an extracted concept. The consistency engine 255 may analyze the information of the PL of the patient and determine whether the extracted concept or a substantially similar diagnosis is present in the PL of the patient. In this manner, the removal operation of the negation engine 240 may provide insight as to whether the PL of the patient has an inconsistency given the information included in the medical document.

The consistency engine 255 may also utilize an automatic approach or a manual approach. When the automatic approach is used, the consistency engine 255 may utilize the most recent information such as the medical document to override any inconsistencies that may have been determined. Thus, the inconsistent information in the PL of the patient may be removed. When the manual approach is used, the consistency engine 255 may prompt the user with one of two options: erase the status of being a removed extracted concept by the negation engine 240 or remove the inconsistent information from the PL of the patient. Based on the input from the user, the consistency engine 255 may perform a corresponding action. Thus, the consistency engine 255 may address any direct inconsistencies between the medical document and the PL of the patient (e.g., the medical document includes that code C10 is not a diagnosis whereas the PL of the patient includes that code C10 is a diagnosis).

The consistency engine 255 may also address any indirect or implied inconsistencies between the medical document and the PL of the patient, particularly given the hierarchical nature of the ICD-10 codes. In another exemplary embodiment in which the consistency engine 255 may be utilized, inconsistencies may be determined through implication utilizing the negation engine 240 as described above but also utilizing the reasoning engine 245. For example, more generic concepts may also be determined to be an inconsistency. If the medical document includes information indicating that the patient is not diagnosed with code C34.90 (malignant neoplasm of unspecified part of unspecified bronchus or lung), the consistency engine 255 may eliminate any inconsistency in the PL of the patient by removing a direct inconsistency (if the PL includes code C34.90) and by removing implied inconsistencies (if the PL includes code C34 indicating a malignant neoplasm of bronchus and lung). In this manner, the reasoning engine 245 may also provide insight as to whether an inconsistency is present between the medical document and the PL of the patient. Thus, the consistency engine 255 may resolve inconsistencies that are implied when one or more codes in the PL of the patient are more generic than a specific code indicated in the medical document as being negated.

It should be noted that the exemplary embodiments may also be configured to incorporate manual updates to the PL of the patient. For example, the practitioner device 120 may be utilized by a practitioner who is not associated with the reporting server 130 and may thus not have access to the services of the reporting server 130. The practitioner device 120 may still query the PL repository 125 and have a requested PL returned. The practitioner device 120 may update the PL and transmit the updated PL for storage in the PL repository 125.

When the PL repository 125 is utilized by practitioner devices who do not utilize the services of the reporting server 130, the PL repository 125 may include further functionalities. For example, the PL may be updated by different practitioner devices and may also be updated by the reporting server 130. The PL repository 125 may include a combination functionality in which updated PLs are analyzed to combine the newly added information into a single updated PL for the patient. In another example, the PL repository 125 may also include a referral functionality in which the updated PLs are packaged and provided to the reporting server 130. Accordingly, the reporting server 130 may utilize substantially similar operations as described above for the medical documents to efficiently combine the information of the different PLs into a single PL for the patient. In this manner, a substantially consistent updating of the PL may still be performed.

In using the above mechanism, the PL may be updated in a variety of manners. In a first example, the extracted concepts from the medical document may be added to the PL of the patient. In a second example, the extracted concepts from the medical document may replace information in the PL of the patient. That is, the PL may have information removed due to an inclusion of an extracted concept from the medical document. In a third example, the extracted concepts from the medical document may remove information in the PL of the patient such as when an inconsistency exists. Accordingly, the medical document may provide information that may entail the PL to be updated through information being added to the PL or removed from the PL.

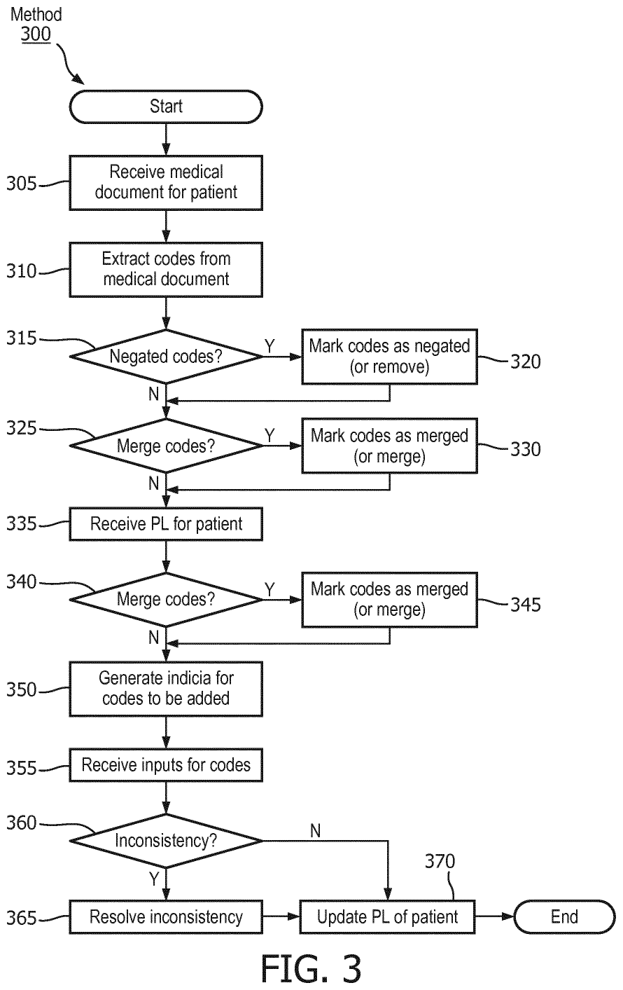

FIG. 3 shows a method 300 for updating a PL of a patient according to the exemplary embodiments. Specifically, the method 300 may relate to the mechanism of the exemplary embodiments in which a medical document is analyzed, concepts are extracted therefrom, and a manner in which the PL of the patient is to be updated using the extracted concepts is determined. Accordingly, the method 300 will be described from the perspective of the reporting server 130. The method 300 will also be described with regard to the system 100 of FIG. 1 and the plurality of engines 235-255 of the reporting server 130 of FIG. 2. The method 300 will further be described with regard to a manual approach of updating the PL of the patient.

In step 305, the reporting server 130 receives a medical document of the patient. As described above, the medical document may be generated in a variety of manners including automatically by a procedure device 105 or manually by a practitioner device 120. The medical document may subsequently be transmitted and stored in a document repository 115. Thus, when information in the medical document is to be included into the PL of the patient, the reporting server 130 may initially query the document repository 115 to have the medical document returned. For example, search parameters including an identification number of the patient may be used to identify any one specific medical document or group of medical documents satisfying a criteria.

In step 310, the reporting server 130 extracts concepts from the medical document. Specifically, the extraction engine 235 analyzes the medical document and determines the information to be considered for inclusion into the PL of the patient. As described above, the extraction engine 235 may utilize any mechanism to extract concepts from the medical document including language parsing mechanisms, code detection mechanisms, etc. The extraction engine 235 may also be configured to correspond the extracted concepts to codes such as ICD-10 codes. Accordingly, the extraction engine 235 may determine a first set of extracted concepts from the medical document. For exemplary purposes, the first set of extracted concepts will be described as a list of ICD-10 codes. However, as described above, the use of ICD-10 codes is only exemplary and the extracted concepts may be other types of codes, other types of identifying diagnoses, prose, etc.

In step 315, the reporting server 130 determines whether any concepts are to be removed or negated from consideration to be included in the PL of the patient. Specifically, the negation engine 240 may determine if any of the extracted concepts in the first set have language or another suggestion indicative of the being removed from consideration within the medical document. As described above, the negation engine 240 may utilize scope detection mechanisms to determine phrases that appear to negate the extracted concept. Thus, if any of the extracted concepts are to be negated, the reporting server 130 continues the method 300 to step 320 where these extracted concepts are marked to be negated or are removed from the first set. After the negation engine 240 has performed its functionality, the first set of extracted concepts may result in a second set of extracted concepts.

In step 325, the reporting server 130 determines whether any concepts within the medical document are to be merged. Specifically, the reasoning engine 245 may determine if any of the ICD-10 codes included in the second set of extracted concepts are more generic or more specific to any other of the ICD-10 codes included in the second set of extracted concepts. That is, an intra-operation is performed. As described above, the ICD-10 codes utilize a hierarchy. Thus, the reasoning engine 245 may utilize a pairwise comparison to determine whether there is any pair of ICD-10 codes that have a specific or generic relationship. Thus, if any of the extracted concepts are to be merged, the reporting server 130 continues the method 300 to step 330 where these extracted concepts are marked to be merged or are merged. After the reasoning engine 245 has performed its functionality, the second set of extracted concepts may result in a third set of extracted concepts.

In step 335, the reporting server 130 may receive the PL of the patient. As described above, the PLs of a plurality of different patients may be stored in the PL repository 125. Accordingly, in a substantially similar manner as having the medical document returned, the reporting server 130 may query the PL repository 125 and have the PL of the patient returned. For example, the identification number of the patient may be used to determine the PL of the patient.

In step 340, the reporting server 130 determines whether any concepts in the medical document are to be merged with any concepts in the PL of the patient. Specifically, the reasoning engine 245 may determine if any of the ICD-10 codes included in the third set of extracted concepts are more generic or more specific to any of the ICD-10 codes included in the PL of the patient. That is, an inter-operation may be performed. Again, the reasoning engine 245 may utilize a pairwise comparison to determine whether there is any pair of ICD-10 codes that have a specific or generic relationship. Thus, if any of the extracted concepts are to be merged with any concept in the PL of the patient, the reporting server 130 continues the method 300 to step 345 where these extracted concepts are marked to be merged or are merged. After the reasoning engine 245 has performed its functionality, the third set of extracted concepts may result in a fourth set of extracted concepts.

In step 350, the reporting server 130 may generate indicia for the extracted concepts in the fourth set. Specifically, the reporting engine 250 may provide a viewing environment in which the user is prompted to enter inputs as to whether each of the extracted concepts in the fourth set are to be included or excluded. For example, an extracted concept having value for inclusion (e.g., more specific to other generic concepts) may be highlighted. In another example, an extracted concept having little value for inclusion (e.g., more generic to other specific concepts) may be lowlighted. In a further example, an extracted concept having a neutral value for inclusion (e.g., no other concept is either more generic or more specific) may utilize regular type face. In step 355, the reporting server 130 may receive the inputs for the extracted concepts in the fourth set having the appropriate visual indicia.

In step 360, the reporting server 130 determines whether there are inconsistencies in the PL of the patient based on the information included in the medical document. As described above, the reporting server 130 may include a further feature to determine inconsistencies, both direct inconsistencies and implied inconsistencies. Specifically, the consistency engine 255 may determine the inconsistencies. The consistency engine 255 may operate in conjunction with the negation engine 240 and/or the reasoning engine 245. In a specific example, the consistency engine 255 may determine the extracted concepts that are marked for removal based upon the functionality of the negation engine 240. Thus, if the consistency engine 255 determines that the PL of the patient already includes the extracted concept, the inconsistency may be determined and removed from the PL of the patient. Accordingly, in step 365, the reporting server 130 resolves any detected inconsistency between the PL of the patient and the medical document. Furthermore, as a manual approach is used, the resolution of the inconsistencies may require the user to enter an input to indicate the manner in which the inconsistency is resolved. For example, the PL of the patient may be updated by removing the inconsistent concept or the negated concept in the medical document may be un-negated.

In step 370, the PL of the patient is updated. Subsequently, the PL of the patient may be displayed for viewing by the user. The PL of the patient may also be transmitted to the PL repository 125 as an updated PL which includes the information of the medical document.

The above method 300 is described with regard to the manual approach. However, as described above, the reporting server 130 may also operate using an automatic approach. Accordingly, the reporting server 130 may not receive any inputs from the user and the reporting server 130, via the appropriate engine, may perform the determinations in an automated manner. For example, all lowlighted concepts may not be included whereas all highlighted and normal type face concepts may be included.

It should be noted that the operations of the reporting server 130 and the updating of the PL of the patient may be performed at a variety of different times. For example, a user may manually activate the PL of the patient to be updated. Specifically, the user may recognize when a medical document associated with the patient has been transmitted to the document repository 115 which the user recognizes may be used to update the PL of the patient. In another example, the reporting server 130 may detect when a medical document has been transmitted to the document repository 115 for the updating operation to be performed. In a further example, the reporting server 130 may perform the PL updating operation at a predetermined time in which one or more medical documents may be analyzed to determine the manner in which the PLs are to be updated.

It should be noted that the above description of the exemplary embodiments is described with updating a PL based on information of a medical document. However, the relation to medicine and documentation in the medical field is only exemplary. The exemplary embodiments may be utilized with any system that updates a document based on information extracted from another document. Thus, the use of the PL and the medical document may represent any scenario in which updating of documentation is performed.

The exemplary embodiments provide a device, system, and method of updating a PL of a patient by analyzing information included in a medical document. A reporting server according to the exemplary embodiments may utilize an automated operation to extract concepts from the medical document and refine the extracted concepts to eliminate unwanted concepts. The reporting server may then include select ones of the extracted concepts into the PL of the patient. In this manner, a consistent mechanism is provided for updating the PL of the patient.

Those skilled in the art will understand that the above-described exemplary embodiments may be implemented in any suitable software or hardware configuration or combination thereof. An exemplary hardware platform for implementing the exemplary embodiments may include, for example, an Intel x86 based platform with compatible operating system, a Windows platform, a Mac platform and MAC OS, a mobile device having an operating system such as iOS, Android, etc. In a further example, the exemplary embodiments of the above described method may be embodied as a computer program product containing lines of code stored on a computer readable storage medium that may be executed on a processor or microprocessor. The storage medium may be, for example, a local or remote data repository compatible or formatted for use with the above noted operating systems using any storage operation.

It will be apparent to those skilled in the art that various modifications may be made in the present disclosure, without departing from the spirit or the scope of the disclosure. Thus, it is intended that the present disclosure cover modifications and variations of this disclosure provided they come within the scope of the appended claims and their equivalent.

What is claimed is:

1. A method, comprising:
at a reporting server:
receiving a first document, the first document associated with a second document, the first document including first information;
extracting first concepts from the first information of the first document;
determining select ones of the first concepts to be included in the second document; and
updating the second document based on the select ones of the first concepts.

2. The method of claim 1, wherein the determining the select ones of the first concepts further comprises:
determining whether one of the first concepts is negated in the first information; and
when the one of the first concepts is negated in the first information, removing the one of the first concepts.

3. The method of claim 1, wherein the determining the select ones of the first concepts further comprises:
determining whether one of the first concepts is merged with another one of the first concepts; and
when the one of the first concepts is merged with the another one of the first concepts, removing one of the one of the first concepts and the another one of the first concepts.

4. The method of claim 3, wherein the first concepts are International Classification of Diseases (ICD) codes.

5. The method of claim 4, wherein the one of the first concepts is a more generic level ICD code.

6. The method of claim 1, wherein the second document includes second concepts.

7. The method of claim 6, wherein the determining the select ones of the first concepts further comprises:
determining whether one of the first concepts is merged with one of the second concepts; and
when the one of the first concepts is merged with the one of the second concepts, removing one of the one of the first concepts and the one of the second concepts.

8. The method of claim 6, wherein the determining the select ones of the first concepts further comprises:
determining whether one of the first concepts is inconsistent with one of the second concepts; and
when the one of the first concepts is inconsistent with the one of the second concepts, removing one of the one of the first concepts and the one of the second concepts.

9. The method of claim 8, wherein the one of the first concepts and the one of the second concepts is one of a direct inconsistency and an implied inconsistency.

10. The method of claim 1, further comprising:
generating an indicia for each of the select ones of the first concepts, a first indicia indicative of a high value for inclusion in the second document, a second indicia indicative of a low value for inclusion in the second document, and a third indicia indicative of a neutral value for inclusion in the second document.

11. A reporting server, comprising:
a transceiver communicating via a communications network, the transceiver configured to receive a first document, the first document associated with a second document, the first document including first information; and
a processor extracting first concepts from the first information of the first document, the processor determining select ones of the first concepts to be included in the second document, the processor updating the second document based on the select ones of the first concepts.

12. The reporting server of claim 11, wherein, for the determining the select ones of the first concepts, the processor determines whether one of the first concepts is negated in the first information, and wherein, when the one of the first concepts is negated in the first information, the processor removes the one of the first concepts.

13. The reporting server of claim 11, wherein, for the determining the select ones of the first concepts, the processor determines whether one of the first concepts is merged with another one of the first concepts, and wherein, when the one of the first concepts is merged with the another one of the first concepts, removing one of the one of the first concepts and the another one of the first concepts.

14. The reporting server of claim 13, wherein the first concepts are International Classification of Diseases (ICD) codes.

15. The reporting server of claim 14, wherein the one of the first concepts is a more generic level ICD code.

16. The reporting server of claim 11, wherein the second document includes second concepts.

17. The reporting server of claim 16, wherein, for the determining the select ones of the first concepts, the processor determines whether one of the first concepts is merged with one of the second concepts, and wherein, when the one of the first concepts is merged with the one of the second concepts, removing one of the one of the first concepts and the one of the second concepts.

18. The reporting server of claim 16, wherein, for the determining the select ones of the first concepts, the processor determines whether one of the first concepts is inconsistent with one of the second concepts, and wherein, when the one of the first concepts is inconsistent with the one of the second concepts, removing one of the one of the first concepts and the one of the second concepts.

19. The reporting server of claim 18, wherein the one of the first concepts and the one of the second concepts is one of a direct inconsistency and an implied inconsistency.

20. A method, comprising:
at a reporting server:
receiving a first document, the first document associated with a second document, the first document including first information;
extracting first concepts from the first information of the first document;
generating a respective prompt for each of the first concepts to be displayed;
receiving a respective input for each prompt;
determining select ones of the first concepts to be included in the second document based on the prompts and the inputs; and
updating the second document based on the select ones of the first concepts.
